SEQUENCE LISTING (39 sequences)

SEQ ID NO: 1 - protein, 9 aa, Human cytomegalovirus
NLVPMVATV

SEQ ID NO: 2 - protein, 5 aa, Artificial Sequence
  linker
GPGPG

SEQ ID NO: 3 - protein, 5 aa, Artificial Sequence
  linker
AAAAA

SEQ ID NO: 4 - protein, 6 aa, Artificial Sequence
  affinity tag
HHHHHH

SEQ ID NO: 5 - protein, 23 aa, Artificial Sequence
  affinity tag
MDYKDHDGDYKDHDIDYKDDDDK

SEQ ID NO: 6 - RNA, 6 nt, Artificial Sequence
  ribosome binding site
aaggag

SEQ ID NO: 7 - RNA, 9 nt, Artificial Sequence
  termination site
uaguaauga

SEQ ID NO: 8 - protein, 397 aa, Artificial Sequence
  human cytomegalovirus pp65 polytope peptide with 6X His affinity tag
MLPHETRLLQTGIHVRVSQPSLILVSQYTPDSGPGPGCPSQEPMSIYVYALPLKMLNIPSINVHHYPSGPGPGVADAVIHASGKQMWQARLTVSGLAWTRQQNQGPGPGQQNQWKEPDVYYTSAFVFPTKDVALRHVVCAGPGPGQVIGDQYVKVYLESFCEDVPSGKLFMHVTLGPGPGTRNPQPFMRPHERNGFTVLCPKNMIIKPGKSGPGPGISHIMLDVAFTSHEHFGLLCPKSIPGLSISGGPGPGTVELRQYDPVAALFFFDIDLLLQRGPQYSEHGPGPGWPPWQAGILARNLVPMVATVQGQNLKYQEFFGPGPGQEFFWDANDIYRIFAELEGVWQPAAQPKRRRGPGPGSIPGLSISGNLLMNGQQIFLEVQAIRETVELHHHHHH

SEQ ID NO: 9 - protein, 9 aa, Human cytomegalovirus
RLLQTGIHV

SEQ ID NO: 10 - protein, 17 aa, Human cytomegalovirus
TGIHVRVSQPSLILVSQ

SEQ ID NO: 11 - protein, 9 aa, Human cytomegalovirus
SIYVYALPL

SEQ ID NO: 12 - protein, 9 aa, Human cytomegalovirus
MLNIPSINV

SEQ ID NO: 13 - protein, 17 aa, Human cytomegalovirus
YALPLKMLNIPSINVHH

SEQ ID NO: 14 - protein, 9 aa, Human cytomegalovirus
QMWQARLTV

SEQ ID NO: 15 - protein, 9 aa, Human cytomegalovirus
YTSAFVFPT

SEQ ID NO: 16 - protein, 9 aa, Human cytomegalovirus
YLESFCEDV

SEQ ID NO: 17 - protein, 16 aa, Human cytomegalovirus
ERNGFTVLCPKNMIIK

SEQ ID NO: 18 - protein, 9 aa, Human cytomegalovirus
IMLDVAFTS

SEQ ID NO: 19 - protein, 16 aa, Human cytomegalovirus
SHEHFGLLCPKSIPGL

SEQ ID NO: 20 - protein, 9 aa, Human cytomegalovirus
ALFFFDIDL

SEQ ID NO: 21 - protein, 9 aa, Human cytomegalovirus
NLVPMVATV

SEQ ID NO: 22 - protein, 9 aa, Human cytomegalovirus
RIFAELEGV

SEQ ID NO: 23 - protein, 9 aa, Human cytomegalovirus
LMNGQQIFL

SEQ ID NO: 24 - protein, 25 aa, Human cytomegalovirus
ETRLLQTGIHVRVSQPSLILVSQYT

SEQ ID NO: 25 - protein, 25 aa, Human cytomegalovirus
QEPMSIYVYALPLKMLNIPSINVHH

SEQ ID NO: 26 - protein, 25 aa, Human cytomegalovirus
AVIHASGKQMWQARLTVSGLAWTRQ

SEQ ID NO: 27 - protein, 25 aa, Human cytomegalovirus
QWKEPDVYYTSAFVFPTKDVALRHV

SEQ ID NO: 28 - protein, 25 aa, Human cytomegalovirus
IGDQYVKVYLESFCEDVPSGKLFMH

SEQ ID NO: 29 - protein, 25 aa, Human cytomegalovirus
FMRPHERNGFTVLCPKNMIIKPGKI

SEQ ID NO: 30 - protein, 25 aa, Human cytomegalovirus
IMLDVAFTSHEHFGLLCPKSIPGLS

SEQ ID NO: 31 - protein, 25 aa, Human cytomegalovirus
LRQYDPVAALFFFDIDLLLQRGPQY

SEQ ID NO: 32 - protein, 25 aa, Human cytomegalovirus
WQAGILARNLVPMVATVQGQNLKYQ

SEQ ID NO: 33 - protein, 25 aa, Human cytomegalovirus
FWDANDIYRIFAELEGVWQPAAQPK

SEQ ID NO: 34 - protein, 25 aa, Human cytomegalovirus
GLSISGNLLMNGQQIFLEVQAIRET

SEQ ID NO: 35 - DNA, 22 bp, Artificial Sequence
  Promoter primer
cttctaatacgactcactatag

SEQ ID NO: 36 - DNA, 104 bp, Artificial Sequence
  Template
tcattactattgaccttgaacagttgcaaccataggtacaagattacgtgcaagcatatttattcctccttatacttaagccctatagtgagtcgtattagaag

SEQ ID NO: 37 - RNA, 83 nt, Artificial Sequence
  Transcript
gggcuuaaguauaaggaggaauaaauaugcuugcacguaaucuuguaccuaugguugcaacuguucaaggucaauaguaauga

SEQ ID NO: 38 - protein, 16 aa, Artificial Sequence
  Epitope
MLARNLVPMVATVQGQ

SEQ ID NO: 39 - RNA, 3 nt, Artificial Sequence
  Start codon
aug

(19) United States
(12) Patent Application Publication
(10) Pub. No.: US 2024/0027462 A1
(43) Pub. Date: Jan. 25, 2024

(54) ARRAYED PEPTIDE NEOEPITOPE GENERATOR

(71) Applicant: NANTBIO, INC., Culver City, CA (US)

(72) Inventors: Peter SIELING, Culver City, CA (US); Kayvan NIAZI, Culver City, CA (US); Clifford Anders OLSON, Culver City, CA (US); Adam D. LAZAR, Culver City, CA (US)

(21) Appl. No.: 18/249,287

(22) PCT Filed: Oct. 8, 2021
(86) PCT No.: PCT/US21/54116

§ 371 Date: Apr. 17, 2023

Related U.S. Application Data
(60) Provisional application No. 63/093,406, filed on Oct. 19, 2020.

(51) Int. Cl. G01N 33/574; A61K 39/00; G01N 33/50

(52) U.S. Cl. CPC G01N 33/57492; A61K 39/464401; A61K 39/4611; A61K 39/4634; G01N 33/505; G01N 2500/10; A61K 2039/5158

(57) ABSTRACT

The invention relates to array-based methods for identification of neoepitope reactive T cells and to compositions produced using such methods. Aspects of the invention relate to rapid and reliable methods to identify neoepitope reactive T cells.

### CROSS-REFERENCE TO RELATED APPLICATIONS

This application claims the benefit of priority under 35 U.S.C. §119(e) to U.S. Provisional Patent Application No. 63/093,406, filed Oct. 19, 2020. The entire disclosure of U.S. Provisional Patent Application No. 63/093,406 is incorporated herein by reference.

### REFERENCE TO A SEQUENCE LISTING

This application contains a Sequence Listing submitted electronically as a text file by EFS-Web. The text file, named “8774-13-PCT_Seq_Listing_ST25”, has a size in bytes of 12,000 bytes, and was recorded on October 8, 2021. The information contained in the text file is incorporated herein by reference in its entirety pursuant to 37 CFR § 1.52(e)(5).

### BACKGROUND

Although treatment of cancer has been improved over the past few decades, most of the cancer treatments in use rely on surgical procedures, radiation and cytotoxic chemotherapeutics, all of which have serious side effects. Within the last few years cancer immune therapies targeting cancer cells with the help of the patient's own immune system have attracted interest.

The foundation of immunology is based on self-nonself discrimination. Most of the pathogens inducing infectious diseases contain molecular signatures that can be recognized by the host and trigger immune responses. Most tumor cells express different types of tumor antigens.

One class of tumor antigens are the tumor associated antigens, i.e. antigens expressed at low levels in normal tissues and expressed at a much higher level in tumor tissue. Such tumor associated antigens have been the target for cancer vaccines for the last decade. However, immunological treatment directed towards tumor associated antigens exhibit several challenges, including that the tumor cells may evade the immune system by downregulating the antigen in question, and that the treatment can lead to toxicities due to normal cell destruction.

Recently, another class of tumor antigens have been identified, the tumor neoantigens or tumor specific-antigens. Tumor neoantigens can arise due to one or more mutations in the tumor genome, leading to a change in the amino acid sequence of the protein in question. Since these mutations are not present in normal tissue, the side-effects of the treatment directed towards the tumor associated antigens may not arise with an immunologic treatment towards tumor neoantigens.

Accordingly, there is a need for methods to identify neoantigens and other tumor associated alterations capable of producing strong activation of the antigen presenting cells that are needed to elicit a strong T cell response.

### BRIEF DESCRIPTION OF THE DRAWINGS

FIG. 1: Workflow diagram of a rapid and reliable method to identify neoepitope reactive T cells from the blood of a cancer patient, involving establishing short-term T-cell lines (TCL) to enrich the population of low-frequency neoepitope-reactive T-cells.

FIG. 2: Synthetic DNA templates for creating neoepitope peptides in vitro.

FIGS. 3A-3D show the T cell response to individual epitopes within pp65 polytope using in vitro transcribed and translated peptides. T cells from short term T cell line generated with pp65 polytope expressed in E. coli, were examined for binding to a soluble human leukocyte antigen (HLA)-A2 protein loaded with a pp65 peptide (sequence NLVPMVATV (SEQ ID NO:1)) to confirm T cells reactive with pp65 (FIG. 3A). The T cells were enriched for the CD8 population using magnetic bead depletion (FIG. 3B). The CD8-enriched T cells were added to monocyte-derived dendritic cells (MoDC) pulsed with peptides transcribed and translated from DNA oligos as described for FIG. 2. FIG. 3C shows the T cell response to all eleven in vitro transcribed and translated peptides as demonstrated to be higher than the response to a control epitope (FLAG). *p<0.05 compared to control epitope (Student's t test). Y-axis units are IFNγ spot forming cells (SFC)/10⁴. FIG. 3D shows the T cell response to three of eleven epitopes (Ep1, Ep6, Ep9) from a T cell line derived from a second HLA-A201, CMV seropositive subject. *p<0.05 compared to control epitope (Student's t test). Y-axis units are IFNy spot forming cells (SFC)/10⁴.

### SUMMARY

In one embodiment, the invention involves an array-based method for identifying neoepitope antigen-reactive T-cells. In some aspects, the invention involves identifying tumor neoepitopes in a sample from a patient wherein nucleotide sequences of the neoepitopes are determined. In some aspects, the method includes expressing in E. coli and purifying a polytope polypeptide of up to 100 linked epitope sequences derived from said nucleotide sequences. In some aspects, patient-derived monocyte-derived dendritic cells (MoDCs) from the patient sample are pulsed with the polypeptide. In some aspects, the invention involves co-culturing the pulsed MoDCs with autologous T-cells, wherein neoepitope antigen reactive T-cells are expanded and later enriched by size via density gradient purification.

In another embodiment, the invention involves pulsing human leukocyte antigen (HLA)-expressing cells matched to the HLA type of the patient with the polypeptide, and in some aspects the invention involves co-culturing the pulsed HLA-expressing cells with autologous T-cells, wherein neoepitope antigen reactive T-cells are expanded and later enriched by size via density gradient purification.

In some aspects, within a support, an array of peptides is produced with the isolated individual neoepitope sequences of the active polytope that has been identified and confirmed as T-cell activating. In some aspects, the invention involves sequentially pulsing the arrayed peptides first with the patient-derived MoDC's, or in other aspects, the HLA-expressing cells, and then with the enriched T-cells. In some aspects, the invention involves confirming the identity (specificity and activity) of the neoepitope antigen-reactive T-cells.

In another embodiment, the invention involves a method of treating a patient comprising. identifying neoepitope antigen-reactive T-cells in the patient, producing a population of neoepitope antigen reactive T-cells involving the use of one or more peptides comprising amino acid sequences identical to the patient-derived neoepitopes, and administering the neoepitope antigen reactive T-cells into the patient.

In another embodiment, the invention involves a method of a method of treating a patient by identifying neoepitope antigen-reactive T-cells in a donor patient, producing a population of neoepitope antigen reactive T-cells involving use of one or more peptides comprising amino acid sequences identical to the donor patient-derived neoepitopes, and administering the neoepitope antigen reactive T-cells into the patient in need thereof.

In some aspects, the sample is a blood sample from the patient.

In some aspects, the support comprises sample reservoirs, wherein each sample reservoir comprises a polypeptide corresponding to one of the tumor neoepitopes.

In some aspects, the polypeptides are produced by a cell-free transcription and translation method.

In some aspects, the amount of the polypeptides is quantified.

In some aspects, the step of confirming the identity of the neoepitope antigen-reactive T-cells includes exposing the enriched neoepitope antigen reactive T cells to an agent capable of identifying antigen reactive T-cells, wherein the agent is peptide-major histocompatibility complex (MHC)-dextramer.

In some aspects, the step of confirming the identity of the neoepitope antigen-reactive T-cells comprises an enzyme-linked immunospot (ELISpot) assay.

In some aspects, the polytope is comprised of tumor associated antigens.

In another embodiment, the invention includes a method of producing an immunotherapeutic comprising antigen-reactive T-cells. In some aspects, the method comprises identifying neoepitope antigen-reactive T-cells. In some aspects, the invention involves producing a population of neoepitope antigen reactive T-cells using one or more peptides that contain amino acid sequences identical to the patient-derived neoepitopes.

### DETAILED DESCRIPTION

The present invention relates to the use of array-based methods for the identification of neoepitope antigen-reactive patient-derived T cells. The aspects of the invention are capable of high-throughput identification of neoepitope antigen-reactive T cells. The use of array-based methods permits much or all of the process to be carried out in a single sample reservoir. For example, a microplate can be used to hold samples in many reservoirs, or wells, and much or all of the identification procedure can be carried out in the microplate, with one sample contained in each well.

### Neoantigens and Neoepitopes

An antigen is generally a substance that induces an immune response. A neoantigen is generally an antigen that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or non-frameshift indel, missense or nonsense substitution, splice site alteration, genomic rearrangement or gene fusion, or any genomic or expression alteration. A mutation can also include a splice variant. Mutations can also affect posttranscriptional and/or posttranslational modifications, and posttranslational modifications that occur in a tumor cell can produce a neoantigen. Post-translational modifications specific to a tumor cell can include aberrant phosphorylation, glycosylation, proteolysis, and/or other posttranslational modifications. Post-translational modifications specific to a tumor cell can also include a proteasome-generated spliced antigen. A tumor neoantigen is generally a neoantigen present in a subject's tumor cell or tissue but not in the subject's corresponding normal cell or tissue. Neoepitopes are the antigenic determinants of neoantigens and are recognized by the immune system. Neoepitopes are recognized by T cells, which can be useful for the immune system-based treatment of cancer.

Tumor associated antigens are antigens that are enriched in tumor cells but are also present, usually at low levels, in non-tumor cells.

### Antigen-Reactive T Cells

Antigen presenting cells (APCs) are cells that display antigens on their surface in the process known as antigen presentation. T-cells can recognize these complexes using their T-cell receptors (TCRs), so APCs may process antigens and present them to T-cells. Examples of APCs include, but are not limited to, dendritic cells (DCs), monocytes, macrophages, certain B-cells, and certain activated epithelial cells.

Dendritic cells are antigen presenting cells that are part of the mammalian immune system. Dendritic cells recognize pathogens and present antigens from those pathogens on the dendritic cell surface for other cells in the immune system. Immature dendritic cells constantly sample foreign antigens from the environment in order to detect pathogens such as viruses and bacteria. This is accomplished by pattern recognition receptors (PRRs), such as CD206. PRRs recognize distinctive chemical moieties that appear in some groups of pathogens, and once they come into contact with pathogens, they become activated mature dendritic cells, and begin to migrate to the lymph nodes. Immature dendritic cells digest pathogens via phagocytosis, break down proteins, and then display their fragments on the cell surface using major histocompatibility complex (MHC). At the same time, they increase the ability to activate T cells by increasing the amount of cell surface receptors such as CD80, CD86 and CD40, which are used as co-receptors in T cell activation. They also induce the migration of dendritic cells into the spleen through the blood vessel or into the lymph node through the lymphatic system by increasing the expression of CCR7. Dendritic cells are therein used as antigen presenting cells to present the antigen of pathogens to helper T cells, cytotoxic T cells (killer T cells), and B cells or activate the cells via non-antigen specific co-stimulatory signals.

Dendritic cells can function in the immune-mediated prevention and response to cancer by presenting cancer associated antigens. For example, cancer-associated dendritic cells play an important role in the T cell-cancer immune response by transporting cancer antigens to the draining lymph node and cross-presenting the cancer antigen to cytotoxic T cells.

Dendritic cells can be derived from monocytes by methods that are well known in the art to produce monocyte-derived dendritic cells (MoDCs).

Dendritic cells assist in recognition of antigens present in tumors by degrading antigens and displaying them for T cells. Tumors contain many mutations and other alterations relative to normal tissue. Dendritic cells can be used to facilitate recognition of tumor cells, by displaying antigens that are specific to or enriched in tumor cells. Dendritic cells can thus facilitate activation of T cells to enable the destruction of tumor cells that contain antigens that are specific to or enriched in the tumor cells. T cells that are capable of destroying or facilitating the destruction of tumor cells are thus useful in methods that involve the use of dendritic cells to activate T cells.

T cells can be used in various ways to treat cancer. T cells can be removed from a subject or patient, modified and then replaced into the patient to facilitate the destruction of tumors. Furthermore, similar to the use of dendritic cells and/or T cells in the destruction of tumor cells, dendritic cells and/or T cells can also facilitate the slowing of tumor cell division, growth, angiogenesis, and/or alter the characteristics of tumor cells.

### Major Histocompatibility Complex (MHC)

There are two primary classes of major histocompatibility complex (MHC) molecules, MHC I and MHC II. MHC I is found on the cell surface of all nucleated cells in the body. One function of MHC I is to display peptides of non-self-proteins from within the cell to cytotoxic T cells. The MHC I complex-peptide complex is inserted into the plasma membrane of the cell presenting the peptide to the cytotoxic T cells, whereby an activation of cytotoxic T cells against the particular MHC-peptide complex is triggered. The peptide is positioned in a groove in the MHC I molecule, allowing the peptide to be about 8-10 amino acids long. MHC II molecules are a family of molecules normally found only on antigen-presenting cells such as dendritic cells, mononuclear phagocytes, some endothelial cells, thymic epithelial cells, and B cells.

As opposed to MHC I, the antigens presented by class II peptides are derived from extracellular proteins. Extracellular proteins are endocytosed, digested in lysosomes, and the resulting antigenic peptides are loaded onto MHC class II molecules and then presented at the cell surface. The antigen-binding groove of MHC class II molecules is open at both ends and is able to present longer peptides, generally between 15 and 24 amino acid residues long.

Class I MHC molecules are recognized by T cell receptors on cells expressing CD8 co-receptors, normally called CD8+cells, whereas class II MHC molecules are recognized by T cell receptors on cells expressing CD4 co-receptors, normally called CD4+cells.

### Identification of Tumor Neoepitopes

The identification of tumor neoepitopes typically involves the comparison of one or more analyses of a tumor sample with a non-tumor sample to identify neoepitopes that are present in the tumor sample but not in the non-tumor sample. Tumor neoepitopes can be identified by many different methods, for example by genomic, transcriptomic, and/or proteomic methods. Typically, tumor neoepitopes, tumor neoantigens, and tumor associated antigens are identified by comparing material present in a tumor sample(s) to material present in a non-tumor sample(s). Typically, the tumor sample and non-tumor sample are derived from the same patient. The material to be compared can be DNA, RNA, protein, or other material present in, on, around, or near cells.

Among other options, it is contemplated that genomic and/or transcriptomic analysis can be performed by any number of analytic methods, however, especially preferred analytic methods include WGS (whole genome sequencing) and exome sequencing of both tumor and matched normal sample using next generation sequencing such as massively parallel sequencing methods, ion torrent sequencing, pyrosequencing, etc. Protoeomic analysis can be performed using various methods of detecting proteins, for example with techniques involving antibodies to recognize specific epitopes and/or antibody-free methods. Antibody-free methods include mass spectrometry, exemplified by MALDI/ToF (matrix-assisted laser desorption/ionization/Time of Flight) and tandem mass spectrometry. Mass spectrometry can also be quantitative, for example with isobaric tags for relative and absolute quantitation (iTRAQ), tandem mass tag (TMT), stable isotope labeling by amino acids in cell culture (SILAC) and/or other quantitative methods.

With respect to filtering identified neoepitopes, it is generally contemplated that neoepitopes are especially suitable for use herein where omics (or other) analysis reveals that the neoepitope is actually expressed. Identification of expression and expression level of a neoepitope can be performed in all manners known in the art and preferred methods include quantitative RNA (hnRNA or mRNA) analysis and/or quantitative proteomics analysis. Most typically, the threshold level for inclusion of neoepitopes will be an expression level of at least 20%, and more typically at least 50% of expression level of the corresponding matched normal sequence, thus ensuring that the (neo)epitope is at least potentially ‘visible’ to the immune system. Consequently, it is generally preferred that the omics analysis also includes an analysis of gene expression (transcriptomic analysis) to so help identify the level of expression for the gene with a mutation. There are numerous methods of transcriptomic analysis known in the art, and all of the known methods are deemed suitable for use herein. For example, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is sometimes considered a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis. Most typically, RNA quantification and sequencing is performed using qPCR and/or rtPCR based methods, although other methods (e.g., solid phase hybridization-based methods) are also deemed suitable. Viewed from another perspective, transcriptomic analysis may be suitable (alone or in combination with genomic analysis) to identify and quantify genes having a cancer and patient specific mutation.

Similarly, proteomics analysis can be performed in numerous manners to ascertain expression of the neoepitope, and all known manners of proteomics analysis are contemplated herein. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity.

In addition, neoepitopes may also be subject to detailed analysis and filtering using predefined structural and/or sub-cellular location parameters. For example, it is contemplated that neoepitope sequences are selected for further use if they are identified as having a membrane associated location (e.g., are located at the outside of a cell membrane of a cell) and/or if in silico structural calculation confirms that the neoepitope is likely to be solvent exposed or presents a structurally stable epitope, etc.

Consequently, it should be recognized that patient and cancer specific neoepitopes can be identified from omics information in an exclusively in silico environment that ultimately predicts potential epitopes that are unique to the patient and tumor type.

Identified and/or predicted neoepitopes may be compared against a database that contains known human sequences to avoid use of a human-identical sequence. Moreover, filtering may also include removal of neoepitope sequences that are due to SNPs in the patient. For example, The Single Nucleotide Polymorphism Database (dbSNP) is a free public archive for genetic variation within and across different species developed and hosted by the National Center for Biotechnology Information (NCBI) in collaboration with the National Human Genome Research Institute (NHGRI). Although the name of the database implies a collection of one class of polymorphisms only (i.e., single nucleotide polymorphisms (SNPs)), it in fact contains a relatively wide range of molecular variation: (1) SNPs, (2) short deletion and insertion polymorphisms (indels/DIPs), (3) microsatellite markers or short tandem repeats (STRs), (4) multinucleotide polymorphisms (MNPs), (5) heterozygous sequences, and (6) named variants. The dbSNP accepts apparently neutral polymorphisms, polymorphisms corresponding to known phenotypes, and regions of no variation. Using such database, the patient and tumor specific neoepitopes may be further filtered to remove the known sequences, yielding a therapeutic sequence set with a plurality of neoepitope sequences.

It should be appreciated that identified cancer neoepitopes can be unique to the patient and the particular cancer in the patient (e.g., having a frequency of less than 0.1% of all neoepitopes, and more typically less than 0.01% in a population of cancer patients diagnosed with the same cancer), Also, the identified cancer neoepitopes can have a high likelihood of being presented in a tumor and therefore provide a high likelihood of being specifically targeted by a synthetic antibody, even if the cancer has an immune suppressive microenvironment.

Neoepitopes can also be filtered for binding affinity to MHC. See, e.g. Lundegaard C. et al. NetMHC-3.0: accurate web accessible predictions of human, mouse and monkey MHC class I affinities for peptides of length 8-11. Nucleic Acids Res 2008;36:W509-12; Lundegaard C. et al. Accurate approximation method for prediction of class I MHC affinities for peptides of length 8, 10 and 11 using prediction tools trained on 9mers. Bioinformatics 2008;24:1397-8.

Binding affinity, and particularly differential binding affinity may also be determined in vitro using various systems and methods. For example, antigen presenting cells of a patient or cells with matched HLA-type can be transfected with a nucleic acid (e.g., viral, plasmid, linear DNA, RNA, etc.) to express one or more neoepitopes using constructs as described in more detail below. Upon expression and antigen processing, the neoepitopes can then be identified in the MHC complex on the outside of the cell, either using specific binders to the neoepitope or using a cell based system (e.g., PBMC of the patient) in which T cell activation or cytotoxic NK cell activity can be observed in vitro.

### Neoepitope and Neoantigen Production

Peptides with the same amino acid sequence as a neoepitope and/or neoantigen can be produced by various methods, for example by heterologous expression in bacteria or with a transcription and translation system. Methods of producing peptides in bacteria are well known in the art, for example with the use of CLEARCOLI® endotoxin-free bacteria (Mamat, U., Woodard, R., Wilke, K. et al. Endotoxin-free protein production—CLEARCOLI® technology. Nat Methods 10, 916 (2013)). Cell free systems, for example transcription and translation (TnT) systems can be used to produce peptides (Chong, S. (2014), Overview of Cell-Free Protein Synthesis: Historic Landmarks, Commercial Systems, and Expanding Applications. Current Protocols in Molecular Biology, 108: 16.30.1-16.30.11). For example, cell free protein expression systems can be produced from E. coli.

Peptide quantitation can be performed using established methods, e.g. measurement of UV absorbance at 280 nm, Bicinchoninic acid (BCA) and Bradford assays. Other types of peptide quantitation, including fluorescent dye methods and other methods, can also be used.

### Polytope Peptides

The neoepitopes of the invention can be incorporated into one or more polytope peptides. A polytope peptide is a peptide with one or more epitopes and/or neoepitopes. Epitope and neoepitope sequences can be between about 6 and about 50 amino acids. Epitope and neoepitope sequences can be at least 8, at least 9, at least 10, at least 11, at least 12, at least 13, at least 14, at least 15, at least 16, at least 17, at least 18, at least 19, at least at least 25, at least 30, at least 35, at least 40, and/or at least 45 amino acids. Epitope and neoepitope sequences can be at most 50, at most 45, at most 40, at most 35, at most 30, at most 25, at most 20, at most 19, at most 18, at most 17, at most 16, at most 15, at most 14, at most 13, at most 12, at most 11, at most 10, at most 9, and/or at most 8 amino acids.

Polytope peptides can include one or more linker sequences that flank and/or interspace the one or more neoepitopes and/or neoantigens of a polytope. Such linker sequences can have amino acids with different properties, for example amino acids the limit rotation and/or introduce two- and/or three-dimensional rigidity to the polypeptide and/or amino acids that allow flexibility. For example, linker sequences can be GPGPG (SEQ ID NO: 2), AAAAA (SEQ ID NO:3), and or other amino acid sequences. Polytope peptides are not required to contain one or more linker sequences.

Polytope peptides can include one or more sequences to facilitate purification. For example, polytope sequences can include one or more affinity tags. For example, polytope peptides can include a 6X His tag (e.g. EIREITIHH (SEQ ID NO:4)).

Polytope peptides can be soluble or insoluble. For example, polytope peptides can be in the form of a suspension of insoluble peptide.

### Rapid and Reliable Identification of Neoepitope- and/or Neoantigen-Reactive T Cells

In some aspects, arrayed peptides are pulsed with patient-derived monocyte-derived dendritic cells (MoDCs) and T cells. Arrayed peptides can be in a support, for example in a reservoir. An array can include a plurality of supports, for example reservoirs, which can be affixed to each other. For example, reservoirs can be the wells of a microplate (i.e. a microtiter plate). A microplate can be a standard laboratory microplate. A microplate can be untreated or treated to facilitate cell adhesion to the microplate or to influence the characteristics of the microplate. A microplate can have 96, 384, 1536, or a different number of wells. Other types of reservoirs, for example cell culture plates, strips of wells, tubes, and/or other types of reservoirs, can also be used to arrange an array.

Peptides corresponding to identified and/or predicted neoepitopes and/or neoantigens are distributed into reservoirs. A peptide corresponding to neoeptiopes and/or neoantigens have the same amino acid sequence as the neoepitope and/or neoantigen to which they correspond. Peptides corresponding to a single neoepitope and/or neoantigen can be placed into a single reservoir, with each reservoir containing peptides that correspond to only one neoepitope and/or neoantigen. Each reservoir can contain only peptides with a single sequence, a pool of peptides corresponding to a single neoepitope and/or neoantigen, and/or a pool of peptides corresponding to multiple neoepitopes and/or neoantigens. MoDCs can be added to the reservoir, for example to pulse the peptides to activate the MoDCs. T cells can also be added to the reservoirs, for example to pulse the peptides and/or MoDCs to facilitate activation of the T cells to recognize the neoepitope and/or neoantigen.

In some aspects, the invention involves enrichment of antigen-specific, or antigen-reactive, T-cells. Enrichment can be done by density gradient centrifugation with Ficoll. Density gradient centrifugation enriches for cells with a density less than the density gradient medium. For example, Ficoll-Paque has a density that exceeds that of water and most live cells. Moreover, activated T cells become larger than unactivated T cells, giving the activated T cells a lower density than unactivated T cells. Density gradient centrifugation can be used to enrich for activated T cells by separating the activated T cells from unactivated T cells.

In some aspects, the T cells are expanded to produce more of and/or a higher frequency of antigen-reactive T cells. For example, T cells that have been enriched in neoepitope and/or neoantigen reactive T cells can be cultured for multiple days. T cells can be expanded for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or another number of days. T cells can be expanded for about 1 week, about 2 weeks, about 3 weeks, about 4 weeks, about 5 weeks, about 6 weeks, or another number of weeks. T cells can be grown in appropriate media, and the media can be supplemented with desired growth factors. For example, T cells can be grown in media supplemented with IL-2, IL-2/7/15, and/or Superkine (SK IL-7/15/21).

Expanded or nonexpanded T cells can be evaluated for the frequency of antigen reactive T cells. The frequency of antigen reactive T cells can be evaluated using a peptide loaded dextramer, for example using HLA-A2 dextramer loaded with one or more peptides that correspond to one or more neoepitopes and/or neoantigens. The frequency of antigen reactive T cells can be evaluated using an ELISPOT assay, for example using an ELISPOT assay with one or more peptides that correspond to one or more neoepitopes and/or neoantigens used to probe for T cells that are reactive against the one or more neoepitopes and/or neoantigens.

By tracking the peptides through the course of the array-based identification method of neoepitope- and/or neoantigen-reactive T cells as disclosed herein, the inventors have made the surprising discovery that they can identify neoepitopes and/or neoantigens that are the most effective for producing neoepitope and/or neoantigen reactive T cells. The sequence of the peptide(s) placed in each reservoir are known, and the identity of the peptides can be tracked through each step in the process because most or all steps occur within the single reservoir. If transfer of materials from one reservoir to another is required, it is possible to track the contents of each reservoir as it is placed into a new reservoir. For example, material from the wells of a microplate can be transferred to same, corresponding wells of another microplate to facilitate easy tracking of the contents of each well or reservoir. By tracking the identity (i.e. the sequence) of each peptide from the original array in which the peptides are placed, through the process, and to the final steps of evaluating the frequency and/or number of reactive t cells, one can identify which peptides produce the strongest effect, in terms of producing reactive T cells.

### Production of a Population of Neoepitope Antigen Reactive T-Cells

The rapid and reliable identification of neoepitope- and/or neoantigen-reactive T cells can be used in the production of T cells for use in therapies, such as immunotherapies. For example, populations of T cells can be split off at various steps in the process of identifying reactive T cells, cultured, and used as an immunotherapeutic. For example, prior to evaluating the frequency of reactive cells, a portion of each reservoir can be split off and cultured separately. The reservoirs that contain sufficient reactive T cells can then direct the user to the appropriate population of T cells that was split off. That involves tracking the identity of the cells in each reservoir, so that the reservoirs with desirable frequencies or numbers of reactive T cells can be matched between the reservoirs that contain the T cells undergoing evaluation and the corresponding populations of T cells that were split off. The array-based methods of the present invention facilitate the tracking of the various T cell populations, enabling the user to know which cells come from T cell populations and reservoirs that are high in the number and/or frequency of T cells that are reactive to the known or identified peptides originally placed in the reservoir(s).

Furthermore, the identity of neoepitopes and/or neoantigens that are the most effective at inducing the production of reactive T cells can be used to produce new populations of neoepitope and/or neoantigen reactive T cells. For example, T cells can be removed from a patient, treated with MoDCs activated with peptides that correspond to neoepitopes and/or neoantigens that have proven to be effective in producing reactive T cells, expanded ex vivo, and then replaced back into the same patient as an immunotherapy. T cells can also be removed from a first or donor patient, treated with MoDCs activated with peptides that correspond to neoepitopes and/or neoantigens that have proven to be effective in producing reactive T cells, expanded ex vivo, and then placed into a second patient as an immunotherapy.

### Identification of Antibodies that Bind T-Cell Activating Peptides and/or Neoepitopes

As described in Example 5, peptides derived from neoepitopes and the processes described herein can be used to screen for monoclonal antibodies. CD16-expressing natural killer cells can be used to facilitate identification of antibodies bound to WIC-presented peptides on the surface of patient-derived MoDCs and/or HLA expressing cells. CD16 is expressed on natural killer cells and certain other cells and binds to immunoglobulins. CD16 binding to immunoglobulins stimulates interferon-y production by natural killer cells. Interferon-γ can then be detected, indicating that the sampled antibody is bound to the peptide of interest.

The terms “peptide” and “polypeptide” are used synonymously herein to refer to polymers constructed from amino acid residues.

The term “amino acid residue” as used herein refers to any naturally occurring amino acid (L or D form), non-naturally occurring amino acid, or amino acid mimetic (such as peptide monomer).

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The following experimental results are provided for purposes of illustration and are not intended to limit the scope of the invention.

### EXAMPLES

### Example 1.

This example describes a rapid and reliable method to identify neoepitope reactive T cells from the blood of a cancer patient.

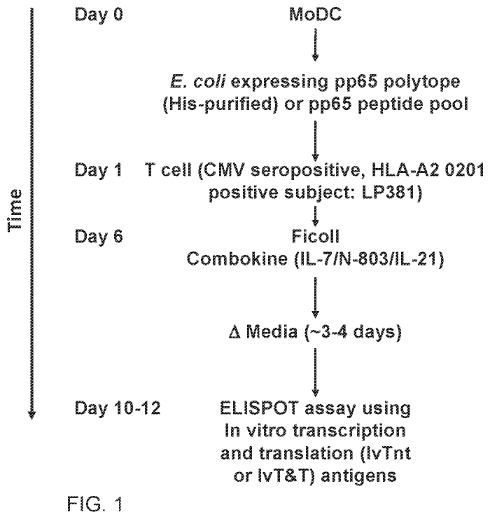

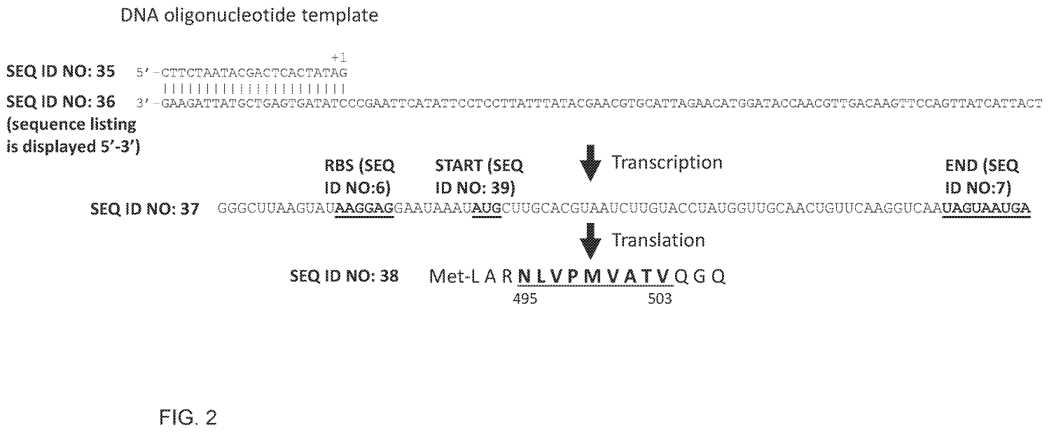

FIG. 1 depicts the workflow of the methods and compositions of this Example. The approach takes advantage of an in vitro transcription and translation system that can generate an antigenic peptide from an oligonucleotide (FIG. 2).

In vitro transcription is efficient using a single stranded oligonucleotide template as long as the promoter is double stranded through +1. The templates include one template strand oligo per epitope plus sense strand universal promoter oligo. The DNA template requires no enzymatic manipulation, purification, cloning: annealed oligos are added to the expression mix for a 2-hour reaction time. Templates include designed control templates (NLV and 3XFlag), CEF class I and II standard peptides and patient specific neoepitopes, IDT ultramer oligos (minimize length and GC content). This involves pp65 NLV epitope (class I) +/−3 amino acids. As shown in the diagram of TnT peptide production of FIG. 2, the template is transcribed into RNA, which is then translated into the desired peptide. For FIG. 2, the single stranded oligonucleotide is specific for NLV peptide. The sense strand is the strand of DNA that has the same sequence as the mRNA, which takes the antisense strand as its template during transcription, and eventually undergoes translation in a protein. The antisense strand is thus responsible for the RNA that is translated into a protein, while the sense strand possesses a nearly identical makeup to that of the mRNA.

Neoepitopes with predicted binding affinities <500 nmol/L were retained for further analysis. SEQ ID NO: 8 is the model neoepitope (CMV pp65, designed for HLA-A2 and HLA-DRB1 0101) in polytope form. NetMHC 3.4 (www.cbs.dtu.dk/services/NetMHC-3.4/; Lundegaard C, et al. NetMHC-3.0: accurate web accessible predictions of human, mouse and monkey MHC class I affinities for peptides of length 8-11. Nucleic Acids Res 2008;36:W509-12; Lundegaard C, et al. Accurate approximation method for prediction of class I MHC affinities for peptides of length 8, 10 and 11 using prediction tools trained on 9mers. Bioinformatics 2008;24:1397-8.) was used to predict neoepitope binding to a specific MHC (major histocompatibility complex) HLA (human leukocyte antigen) allele. The top ten predicted epitopes for HLA-A2 binding (Table 1) and the top four epitopes for HLA-DRB1 0101 (Table 2) were encoded in the polytope. In some cases, the epitopes overlapped; thus a total of eleven epitopes (Epl through Epl 1) were encoded within the polytope (Table 3). In the amino acid sequence of the entire polytope (SEQ ID NO: 8), 3 lmer epitopes are separated by five amino acid linkers with the sequence GPGPG (SEQ ID NO:2). A six-His tag is encoded on the C terminus of the polytope to allow for purification.

(SEQ ID NO: 8)
MLPHETRLLQTGIHVRVSQPSLILVSQYTPDSGPGPGCPSQEPMSIYVY
ALPLKMLNIPSINVHHYPSGPGPGVADAVIHASGKQMWQARLTVSGLAW
TRQQNQGPGPGQQNQWKEPDVYYTSAFVFPTKDVALRHVVCAGPGPGQV
IGDQYVKVYLESFCEDVPSGKLFMHVTLGPGPGTRNPQPFMRPHERNGF
TVLCPKNMIIKPGKSGPGPGISHIMLDVAFTSHEHFGLLCPKSIPGLSI
SGGPGPGTVELRQYDPVAALFFFDIDLLLQRGPQYSEHGPGPGWPPWQA
GILARNLVPMVATVQGQNLKYQEFFGPGPGQEFFWDANDIYRIFAELEG
VWQPAAQPKRRRGPGPGSIPGLSISGNLLMNGQQIFLEVQAIRETVELH
HHHHH

TABLE 1
Predicted Binding Affinity (HLA-A2 0201 epitopes)

| | Sequence | Affinity (nM) |
|---|---|---|
| SEQ ID NO: 21 | NLVPMVATV | 29 |
| SEQ ID NO: 15 | YTSAFVFPT | 33 |
| SEQ ID NO: 22 | RIFAELEGV | 34 |
| SEQ ID NO: 23 | LMNGQQIFL | 44 |
| SEQ ID NO: 12 | MLNIPSINV | 54 |
| SEQ ID NO: 14 | QMWQARLTV | 64 |
| SEQ ID NO: 9 | RLLQTGIHV | 66 |
| SEQ ID NO: 11 | SIYVYALPL | 79 |
| SEQ ID NO: 20 | ALFFFDIDL | 82 |
| SEQ ID NO: 18 | IMLDVAFTS | 85 |
| SEQ ID NO: 16 | YLESFCEDV | 133 |

TABLE 2
Predicted Binding Affinity (HLA-DRB1 0101
epitopes)

| | Sequence | Affinity (nM) |
|---|---|---|
| SEQ ID NO: 10 | TGIHVRVSQPSLILVSQ | 5.6 |
| SEQ ID NO: 19 | SHEHFGLLCPKSIPGL | 5.6 |
| SEQ ID NO: 17 | ERNGFTVLCPKNMIIK | 7.5 |
| SEQ ID NO: 13 | YALPLKMLNIPSINVHH | 7.6 |

TABLE 3
IvT&T peptides: Met-25 mer-GKCCPGCC

| | Epitope | Sequence |
|---|---|---|
| SEQ ID NO: 24 | Ep1 | ETRLLQTGIHVRVSQPSLILVSQYT |
| SEQ ID NO: 25 | Ep2 | QEPMSIYVYALPLKMLNIPSINVHH |
| SEQ ID NO: 26 | Ep3 | AVIHASGKQMWQARLTVSGLAWTRQ |
| SEQ ID NO: 27 | Ep4 | QWKEPDVYYTSAFVFPTKDVALRHV |
| SEQ ID NO: 28 | Ep5 | IGDQYVKVYLESFCEDVPSGKLFMH |
| SEQ ID NO: 29 | Ep6 | FMRPHERNGFTVLCPKNMIIKPGKI |
| SEQ ID NO: 30 | Ep7 | IMLDVAFTSHEHFGLLCPKSIPGLS |
| SEQ ID NO: 31 | Ep8 | LRQYDPVAALFFFDIDLLLQRGPQY |
| SEQ ID NO: 32 | Ep9 | WQAGILARNLVPMVATVQGQNLKYQ |
| SEQ ID NO: 33 | Ep10 | FWDANDIYRIFAELEGVWQPAAQPK |
| SEQ ID NO: 34 | Ep11 | GLSISGNLLMNGQQIFLEVQAIRET |

T cells (isolated from peripheral blood) with reactivity to neoepitopes might be present at a frequency that is detectable directly from the blood using this approach. Alternatively, the neoepitope reactive T cells may be in such a low frequency in the blood that they may require expansion before testing in the in vitro transcription/translation system. In this case, a strain of E. coli that lacks LPS as a vector for the neoepitopes can be used.

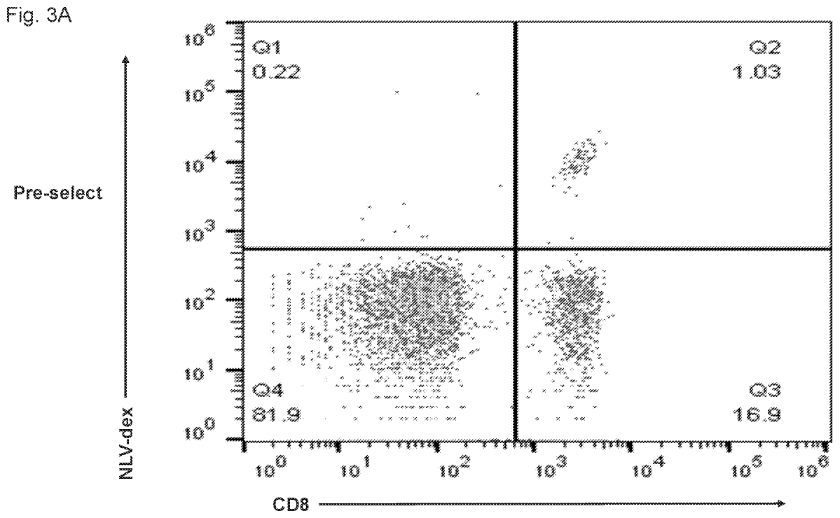

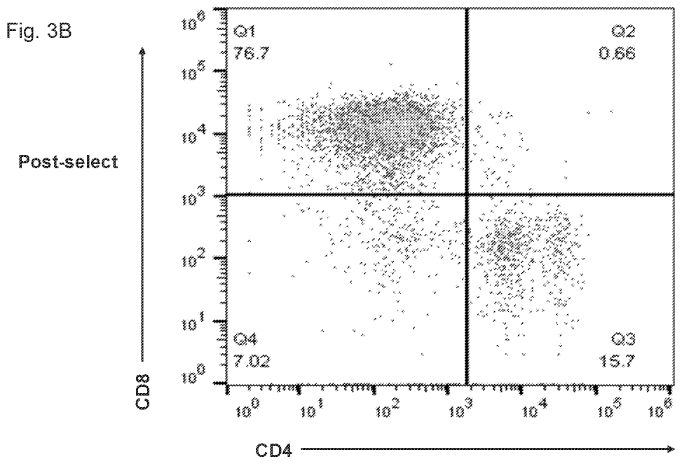

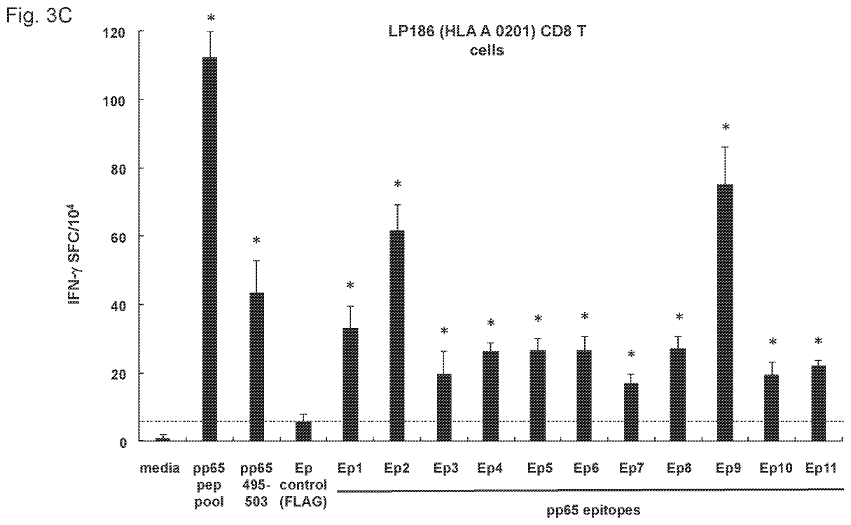

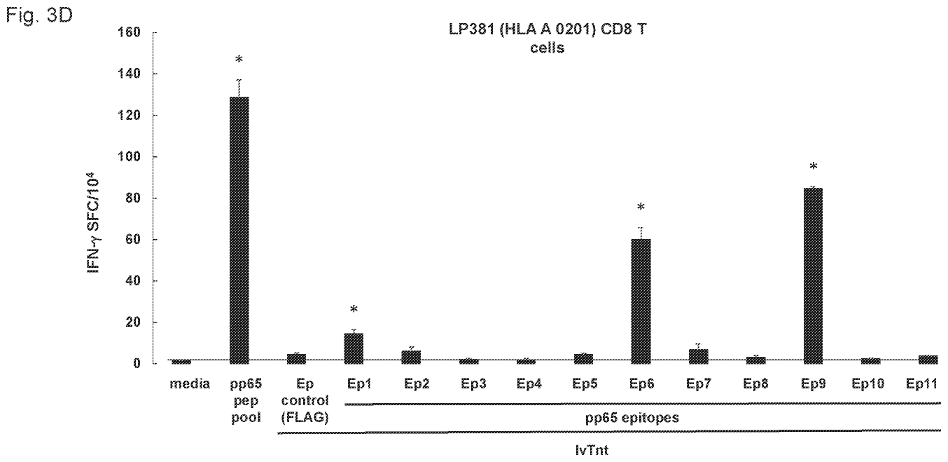

A model cytomegalovirus (CMV) antigen (CMV pp65) was used to test the expansion of T cells from the blood of a healthy donor using pp65 cloned into LPS-deficient E. coli (i.e. CLEARCOLI®, or CC). Monocyte-derived dendritic cells (MoDCs), derived from the blood of a healthy HLA-A2+ subject were pulsed with peptide or a pp65 polytope (SEQ ID NO: 8) purified from CC using the His tag (overnight at 37° C.). Unbound peptide and polytope were washed from the MoDCs, then autologous T cells were combined with the antigen-pulsed MoDCs for 5 days at 37° C. Antigen-specific T cells were enriched using a density gradient solution and centrifugation, then cells were cultured with a cocktail of IL-2/7/15. Cells were cultured for an additional four to six days, then evaluated for antigen reactive T cells using a peptide loaded dextramer (HLA-A2 loaded with pp65 peptide 495-503, also known as NLV peptide) or control dextramer without peptide. FIGS. 3A and 3B show the results of flow cytometry analyses for antigen reactive T cells treated with a peptide loaded dextramer (loaded with NLV peptide) or control dextramer without peptide, along with an anti-CD8 antibody to identify the frequency of CD8 T cells reactive with NLV.

Upon validation that NLV-reactive T cells were present in the culture, CD8 T cells were enriched using magnetic bead selection. Enriched CD8 T cells were identified in FIG. 3B. The CD8 enriched T cells were evaluated for antigen reactive T cells using ELISPOT with the antigen produced by the in vitro transcription and translation system described for FIG. 2. The results of the ELISPOT assay are shown in FIGS. 3C and 3D, meaning detection of pp65 epitope reactive T cells were made using two independent subjects. The epitopes identified by the method (subject LP381, FIG. 3D) are SEQ ID Nos. 9, 21, and 29.

### Example 2.

This Example describes methods similar to those of Example 1, but with one epitope, or neoepitope, per well. Neoepitopes can be identified, selected, and/or designed as in Example 1 or other methods described herein. Neoepitopes can be produced by various methods, including by standard protein production and purification techniques involving E. coil cells and/or cell free translation as described herein, or neoepitopes can be synthesized.

Each neoepitope can be encoded by a plasmid, with each plasmid encoding one neoepitope. One plasmid encoding one neoepitope can be placed in each reservoir of an array, for example in each well of a microplate. The protocols described in Example 1 and elsewhere herein can then be used to identify antigen reactive T cells.

### Example 3.

This Example describes a further application of the methods described in Examples 1 and 2 and elsewhere herein, in which the neoepitope reactive T-cells are processed into a therapeutic reagent for reinsertion back into a patient. T cells can be removed from a patient or another individual (a donor), processed, and returned to the patient in need thereof.

The methods described in Examples 1 and 2 and elsewhere herein can produce antigen reactive T cells that are specific to one or more epitopes, which can be in a polytope. In general, polytopes are generated that contain epitopes predicted to be useful for generating antigen reactive T cells. The methods described herein are used to produce antigen reactive T cells. The generated antigen reactive T cells are then cultured under conditions suitable for producing T cells that can be implanted into a patient. The T cells can be cultured to increase in number. The produced antigen reactive T cells can then be implanted into the patient to produce the desired result.

### Example 4

This Example describes a further application of the methods described in Examples 1 and 2 and elsewhere herein, in which either patient-derived MoDC or an HLA-matched cell line are pulsed with a neoepitope identified as activating patient-derived T-cells by the method of Example 1, wherein the MoDC and T-cells are derived from separate HLA matched patients. This method assumes common neoepitopes between distinct patients. Thus T-cell activating MHC-presented neoepitopes derived from one patient may effectively stimulate T-cells in a separate HLA-matched patient with the same neoepitopes. The invention further contemplates a kit with one or more HLA-denuded cell lines, wherein each cell line is genetically modified to express a unique HLA protein for use with HLA-matched patient-derived neoepitope peptide obtained by the method of Example 1.

### Example 5

This Example describes a further application of the methods described in Examples 1 and 2 and elsewhere herein, in which T-cell activating MHC presented peptides derived from individual epitopes arrayed on a support are used to screen for monoclonal antibodies that bind to those peptides. MoDC or HLA-expressing cell lines are pulsed with identified T-cell activating neoepitopes. Antibody libraries arrayed on a support can be screened against neoepitope-pulsed MoDC or HLA-expressing cell lines. Antibodies bound to MHC- presented peptides on the surface of patient-derived MoDC or an HLA expressing cell line may be identified by the further addition of CD16-expressing natural killer cells, or a CD16-expressing cell line such as NK-92, which would stimulate the production of IFN-y and thus enable detection via ELISA.

### Example 6

This Example describes another application of the methods described in Examples 1 and 2 and elsewhere herein, in which T-cell activating MHC presented peptides derived from individual epitopes arrayed on a support are used to screen for soluble T cell receptors (TCRs), which are not membrane-bound or -embedded, that recognize the peptides. MoDC or HLA-expressing cell lines are pulsed with identified T-cell activating neoepitopes. TCR libraries arrayed on a support can be screened against neoepitope-pulsed MoDC or HLA-expressing cell lines. TCRs bound to MHC-presented peptides on the surface of patient-derived MoDC or an HLA expressing cell line may be detected by various methods, including anti-TCR antibodies, fluorescently labelled TCRs, and/or TCRs fused to fluorescent proteins. For example, anti-TCR antibodies that are bound to TCRs can be then detected by the further addition of CD16-expressing natural killer cells, or a CD16-expressing cell line such as NK-92, which would stimulate the production of IFN-y and thus enable detection via ELISA. Alternatively, anti-TCR antibodies can be fluorescently labelled, TCRs can be fluorescently labelled, and/or TCRs can be fused to a fluorescent protein (e.g. green fluorescent protein (GFP), yellow fluorescent protein (YFP), tdTomato, DsRed, mCherry, or various other fluorescent proteins known in the art) to enable detection.

Unless otherwise specified, it is to be understood that each embodiment of the invention may be used alone or in combination with any one or more other embodiments of the invention.

Variations, modifications, and other implementations of what is described herein will occur to those of ordinary skill without departing from the spirit and the scope of the invention. Accordingly, the invention is not to be limited only to the preceding illustrative description.

Those skilled in the art will recognize or be able to ascertain using no more than routine experimentation, numerous equivalents to the compounds, compositions, and methods of use thereof described herein. Such equivalents are considered to be within the scope of the invention.

The contents of all references, patents and published patent applications cited throughout this Application, as well as their associated figures are hereby incorporated by reference in their entirety. In case of conflict, the present specification, including its specific definitions, will control.

What is claimed is:

1. An array-based method for identifying neoepitope antigen-reactive T-cells comprising:
a. identifying tumor neoepitopes in a sample from a patient wherein nucleotide sequences of the neoepitopes are determined;
b. expressing in E. coli and purifying a polytope polypeptide of up to 100 linked epitope sequences wherein the polytope polypeptides are encoded by one or more expression plasmids derived from said nucleotide sequences;
c. pulsing patient-derived monocyte dendritic cells (MoDCs) from the patient with the polypeptide from step b;
d. co-culturing the pulsed MoDCs with autologous T-cells, wherein neoepitope antigen reactive T-cells are expanded and later enriched by size via density gradient purification;
e. producing within a support, an array of peptides comprising the isolated individual neoepitope sequences of the active polytope identified in step a and confirmed as T-cell activating in step d;
f. sequentially pulsing the arrayed peptides first with the patient-derived MoDC's and then with the enriched T-cells from step d; and
g. confirming the specificity and activity of the neoepitope antigen-reactive T-cells.

2. The method of claim 1, wherein the sample is a blood sample from the patient.

3. The method of claim 1, wherein the support comprises sample reservoirs, wherein each sample reservoir comprises a polypeptide corresponding to one of the tumor neoepitopes.

4. The method of claim 1, wherein alternatively the polypeptides of step b are produced by a cell-free transcription and translation method.

5. hod of claim 1, wherein the amount of the polypeptide of step b is quantified.

6. The method of claim 1, wherein the step of confirming the identity of the neoepitope antigen-reactive T-cells comprises exposing the enriched neoepitope antigen reactive T cells to an agent capable of identifying antigen reactive T-cells, wherein the agent is a peptide-MIIC-dextramer.

7. The method of claim 1, wherein the step of confirming the identity of the neoepitope antigen-reactive T-cells comprises an enzyme-linked immunospot (ELISpot) assay.

8. The method of claim 1, wherein the polytope is comprised of tumor associated antigens.

9. A method of producing an immunotherapeutic comprising antigen-reactive T-cells, wherein the method comprises:
a. identifying neoepitope antigen-reactive T-cells by the method of claim 1; and
b. producing a population of neoepitope antigen reactive T-cells comprising use of one or more peptides comprising amino acid sequences identical to the patient-derived neoepitopes.

10. A method of treating a patient comprising:
a. identifying neoepitope antigen-reactive T-cells in the patient by the method of claim 1;
b. producing a population of neoepitope antigen reactive T-cells comprising use of one or more peptides comprising amino acid sequences identical to the patient-derived neoepitopes; and
c. administering the neoepitope antigen reactive T-cells of step b into the patient.

11. A method of treating a patient in need thereof comprising:
a. identifying neoepitope antigen-reactive T-cells in a donor patient by the method of claim 1;
b. producing a population of neoepitope antigen reactive T-cells comprising use of one or more peptides comprising amino acid sequences identical to the donor patient-derived neoepitopes; and
c. administering the neoepitope antigen reactive T-cells of step b into the patient in need thereof.

12. An array-based method for identifying neoepitope antigen-reactive T-cells comprising:
a. identifying tumor neoepitopes in a sample from a patient wherein nucleotide sequences of the neoepitopes are determined;
b. expressing in E. coli and purifying a polytope polypeptide of up to 100 linked epitope sequences wherein the polytope polypeptides are encoded by one or more expression plasmids derived from said nucleotide sequences;
c. pulsing HLA-expressing cells matched to the HLA type of the patient with the polypeptide from step b;
d. co-culturing the pulsed HLA-expressing cells with autologous T-cells, wherein neoepitope antigen reactive T-cells are expanded and later enriched by size via density gradient purification;
e. producing within a support, an array of peptides comprising the isolated individual neoepitope sequences of the active polytope identified in step a and confirmed as T-cell activating in step d;
f. sequentially pulsing the arrayed peptides first with the HLA-expressing cells and then with the enriched T-cells from step d; and
g. confirming the specificity and activity of the neoepitope antigen-reactive T-cells.

13. The method of claim 12, wherein the sample is a blood sample from the patient.

14. The method of claim 12, wherein the support comprises sample reservoirs, wherein each sample reservoir comprises a polypeptide corresponding to one of the tumor neoepitopes.

15. The method of claim 12, wherein alternatively the polypeptides of step b are produced by a cell-free transcription and translation method.

16. The method of claim 12, wherein the amount of the polypeptide of step b is quantified.

17. The method of claim 12, wherein the step of confirming the identity of the neoepitope antigen-reactive T-cells comprises exposing the enriched neoepitope antigen reactive T cells to an agent capable of identifying antigen reactive T-cells, wherein the agent is a peptide-MHC-dextramer.

18. The method of claim 12, wherein the step of confirming the identity of the neoepitope antigen-reactive T-cells comprises an enzyme-linked immunospot (ELISpot) assay.

19. The method of claim 12, wherein the polytope is comprised of tumor associated antigens.

20. A method of producing an immunotherapeutic comprising antigen-reactive T-cells, wherein the method comprises:
a. identifying neoepitope antigen-reactive T-cells by the method of claim 12; and
b. producing a population of neoepitope antigen reactive T-cells comprising use of one or more peptides comprising amino acid sequences identical to the patient-derived neoepitopes.

21. A method of treating a patient comprising:
a. identifying neoepitope antigen-reactive T-cells in the patient by the method of claim 12;
b. producing a population of neoepitope antigen reactive T-cells comprising use of one or more peptides comprising amino acid sequences identical to the patient-derived neoepitopes; and
c. administering the neoepitope antigen reactive T-cells of step b into the patient.

22. A method of treating a patient in need thereof comprising:
a. identifying neoepitope antigen-reactive T-cells in a donor patient by the method of claim 12;
b. producing a population of neoepitope antigen reactive T-cells comprising use of one or more peptides comprising amino acid sequences identical to the donor patient-derived neoepitopes; and
c. administering the neoepitope antigen reactive T-cells of step b into the patient in need thereof.
